(19) United States
(12) Patent Application Publication
(10) Pub. No.: US 2016/0287493 A1
(43) Pub. Date: Oct. 6, 2016

(54) CLAY BASED PRODUCT IN SHEET FORM FOR COSMETIC APPLICATIONS

(71) Applicant: L'OREAL, Paris (FR)

(72) Inventors: Gaurav AGARWAL, Jodhpur Rajasthan (IN); Jean-Pascal HIRT, Saint Cloud (FR)

(21) Appl. No.: 15/037,836

(22) PCT Filed: Nov. 20, 2014
(86) PCT No.: PCT/EP2014/075195

§ 371 Date: May 19, 2016

(30) Foreign Application Priority Data
Nov. 22, 2013 (IN) 3420/DEL/2013

(51) Int. Cl. A61K 8/26; A61Q 19/00; A61K 8/46; A61K 8/02; A61K 8/73

(52) U.S. Cl. CPC A61K 8/26; A61K 8/0212; A61K 8/731; A61K 8/463; A61Q 19/008

(57) ABSTRACT

The invention relates to an applicator body, wherein said applicator body composition comprises at least one clay and at least one water soluble polymer. The invention also relates to methods for preparing said applicator body and methods of cosmetic care.

The present invention relates to clay based formulation for application on the skin. More particularly the invention relates to applicator body, and for example face masks.

Oiliness of skin is caused by excess secretion of oily/waxy matter from sebaceous gland. Sebaceous gland is found in greatest abundance on the face and scalp, though they are distributed throughout all skin sites except the palms and soles. Sebum contains triglycerides, waxes, squalene and metabolytes of fat-producing cells. Although sebum keeps hair and skin supple, excess sebum can contribute to concentrating of horny cells at the opening of sebaceous follicle on the surface of the skin, this causes constriction in the follicular opening leading to a plug or comedones. This can result in black head, white heads, significant irritation, redness, tissue damage and acne.

Sebum secretion is mainly controlled by dihydrotestosterone (sexually related harmon) and vary in depending of type of skin. There are number of products in a market which claims the oil control properties of the product and uses active such as perlite, mineral clay such as hectorite, bentonite, and quaternized derivatives thereof.

The use of mineral clays as medicinal and cosmetic tool is popular since a long time.

Clays are used to absorb excess oil, dirt, and toxins from the skin while simultaneously exfoliating and improving skin circulation. Some clays, such as bentonite clay, are primarily ingested for medicinal purposes such as detoxification or mineral deficiencies.

Other clays, such as French Green clay and Rhassoul clay, are used externally for skin conditions and for cosmetic purposes. Clays come in a variety of colors such as red, green, white, gray, and can range in texture from coarse and heavy to fine and fluffy. The different colors of clays occur because of their natural mineral content.

In modern cosmetics, such clays are used as key active ingredient for oil control products are sold in the form of oil control face wash etc. For example: US patent 2006/0292108 describes skin care composition composed of 10% Kaolin. Patent WO 93/08793 relates to skin cleansing composition which comprises 3 to 60% by weight of one or more absorbent to absorb soil, grease and oils from skin, the example of absorbent materials given are fuller's earth, china clay, bentonite and mixtures thereof. Ponds clear balance pore tightening facial foam from Unilever, Garnier light 3-in-1 fairness face wash from L'oreal, Naturgo men's clay face wash, Tjoy mineral cleansing foam are some commercial examples of clay based cosmetic products.

Also clay in powder form or in readymade paste form is sold as clay mask or as face pack. The term “Face pack” here refers to a product in the form of a cream or paste which is applied as mask by making a thin layer of the product on skin for specific period of time or till drying of the product and then is removed by rubbing or washed away by water.

Korean patent Laid-open publication No. 10-2005-0062473 discloses a jellied pack containing yellow soil. Korean patent Laid-open publication No. 10-1999-0047642 discloses a cosmetic loess pack and method for manufacturing the same. Non-limiting examples of such commercial products are Argiletz red clay face mask in France, Borghese Fango Brillinate brightening mud mask in Italy, Swanson Ultra Bavarian mountain mud mask in Germany, Moor Krauter packing facial pack in Austria, Blue Lagoon—silica mud mask in Iceland, Handy Okinawa kucha pack in Japan, Yue Sai white ling long instant brightening mask in china, Beautyplus seaweed mask mud mask in China, Ciracle Jeju volcanic clay mask in Korea, Hesh multani mati (Fuller's earth) face pack in India, Ovajan, Npato, mineral mud face & body mask in USA, Calistoga mud mask in USA, Arctic mineral mud mask in Canada, Amazon Rainforest facial mud mask in Brazil.

The application of clay based face wash product are used as rinse-off products, but the oil absorbing efficacy of clay is not fully utilized and therefore such products are not as effective as clay based mask. Clay based mask are applied on the skin in paste form till the drying of clay. The application of such product is a messy process and consumers need to rub the skin for removal of such product. Upon drying of such clay products, when consumer moves his/her facial muscles, dried mud tends to fall down from the face and contaminates bathroom, clothes causing the user to clean or to wash. In general post application of such clay mask, consumers also need to use a cleansing product to clean the face.

The invention aims to solve the technical problem of providing a clay based product which can facilitate easy application and removal for the user.

The invention aims to solve the technical problem of providing a product having similar or greater efficacy than topical clay based products, or at least a satisfying efficacy for the user as clay product.

The invention aims to solve the technical problem of preparing a product which can provide dual functionality of peel off and cleansing where peel off refers to a product which comes off from the skin and cleansing refers to cleaning of skin with a cleansing compound such as soap or surfactant.

The invention aims to solve the technical problem related to drying of clay products, such as fall down of dried mud from the face and contaminating bathroom and/or clothes causing the user to clean or to wash.

The invention aims to solve the above technical problems by providing a single product, optionally made of two pieces.

The invention aims to solve the above technical problems by providing a product and process which is costs saving, industrial and safe for the user.

The present invention provides a solution to the above recited technical problems.

The present invention relates to an applicator body composition comprising at least one clay and at least one water soluble polymer.

The term “clay” refers to a naturally occurring material composed primarily of fine-grained minerals, which is generally plastic at appropriate water contents and will harden with dried or fired. Although clay usually contains phyllosilicates, it may contain other materials that impart plasticity and harden when dried or fired. Associated phases in clay may include materials that do not impart plasticity and organic matter. A common definition is that in the Penguin Dictionary of Science, namely “finely divided rock materials whose component minerals are various silicates, mainly of magnesium or aluminium”. Clay comprises Kaolinite (typically defined as [Si₄]Al₄O₁₀(OH)₈.nH₂O (n=0 or 4)), Illite (typically defined as Mₓ[Si_{6.8}Al_{1.2}]Al₃Fe.025Mg_{0.7}5O20 (OH)₄), Vermiculite (typically defined as .Mₓ[Si₇Al]AlFe.05Mg0.5O₂0 (OH)₄), Smectite (typically defined as Mₓ[Si₈]Al_{3.2}Fe_{0.2}Mg_{0.6}O₂0 (OH)₄, Chlorite (typically defined as (Al(OH)_{2.55})4[Si_{6.8}Al0_{1.2}}Al_{3.4}Mg_{0.6})20(OH)₄).

Another definition, frequently used by chemists is “a naturally occurring sediment or sedimentary rock composed of one or more minerals and accessory compounds, the whole usually being rich in hydrated aluminum silicate, iron or magnesium, hydrated alumina, or iron oxide, predominating in particles of colloidal or near-colloidal size, and commonly developing plasticity when sufficiently pulverized and wetted” (see Kirk-Othmer, Encyclopaedia of Chemical Technology, Volume 5, page 544, 2nd edition, John Wiley and Sons, Inc., New York, N.Y. 1964). Example of clays are given in the book “Clay mineralogy, S. Caillère, S. Henin, M. Rautureau, 2nd edition 1982, Masson.”. Clays may be of natural or synthetic origin.

Hydrophilic clay includes smectites such as saponites, hectorites, montmorillonites, bentonites, beidellite. Hydrophilic clay includes synthetic hectorites (also called laponites) as the products sold by the company under the name Laporte Laponite XLG, Laponite RD, Laponite RDS (these products are sodium silicates and magnesium silicates in particular sodium, lithium and magnesium) bentonites as the product sold under the name Bentone® HC Rheox, magnesium silicates and aluminum products such as hydrated sold by Vanderbilt Company as ultra Veegum®, Veegum® HS, Veegum® DGT, or calcium silicates and particularly the synthetic form sold by the company under the name Micro-Cel® C.

Fuller's earth consists chiefly of hydrated aluminum silicates that contain metal ions such as magnesium, sodium, and calcium within their structure. Montmorillonite is the principal clay mineral in fuller's earth, but other minerals such as kaolinite, attapulgite, and palygorskite among other components.

Lipophilic clay means clay swellable in a lipophilic medium, the clay swells and forms a colloidal dispersion. As lipophilic clays include modified clays such as the modified magnesium silicate (Bentone gel VS38 from Rheox) hectorites modified with an ammonium chloride fatty acid C₁₀ to C₂₂, as hectorite modified with ammonium chloride distearyldimethylammonium (CTFA name: Disteardimonium hectorite) sold under the name “Bentone 38 CE” by Rheox or Bentone® 38V by ELEMENTIS.

The origin of such clay can be natural or synthetic mineral clay such as hectorite, bentonite, and quaternized derivatives thereof, for example which are obtained by reacting the minerals with a quaternary ammonium compound, such as stearalkonium bentonite, hectorites, quaternized hectorites such as Quaternium-18 hectorite, carbonates such as propylene carbonate, bentones, and the like.

The non-limiting of examples of clay which can be used in the present invention are Fuller's earth, Pinatubo volacanic ash mud in Philippines, Aleppo clay from Syria, Pulau tiga volcano mud from Malasiya, Nha Trang mud from Vietnam, White Kaolinite from Korea, Yellow Loess from Korea, Jeju volcanic clay from Korea, Guanziling mud form Taiwan, Wudalianchi volcanic mud from China, Black mud of Yuncheng salt lake from China, mineral mud from Tantou village in China, China clay (Kaolin), Maifan stone from China, Beppu onsen Fango from Japan, Kucha from Japan, Tanakura clay from Japan, Cambrian blue clay from Russia, Blue Lagoon mud from Iceland, Saki lake mud from Ukraine, Karlovy vary moor mud from Czech Republic, Heviz Georgikon moor mud from Hungry, Alpine moor mud from Austria, Bad wilsnack mud from Germany, Bavarian mineral slat mountain mud from Germany, Freiburg volcanic ash from Germany, Santorini mud from Greece, Mar menor mud from Spian, Ischian volcanic mud from Italy, Euganean thermal mud from Italy, Yellow clay-Illite from France, French Green Clay—Montmorrillonite, Calistoga mud from USA, Sacred clay and ormalite from USA, Redmond clay from USA, Arctic mineral mud from Canada, Tulum Mayan clay from Mexico, Glacial clay from Canada, Amazonian white clay from Brazil, El Chillante volcanic thermal mud from Argentina, African healing clay, Australian olive green clay.

A second essential component of the composition is at least one water soluble polymer.

Advantageously, a water soluble polymer is selected among water soluble polymers. More preferably this is a filmogenic polymer. A filmogenic polymer is adapted to form alone or with other filmogenic agents, a continuous film, preferably adhering to skin.

By “water soluble polymer” is meant that applicator body made of at least this polymer, upon immersion in water at 25° C. disintegrate and/or dissolve slowly e.g. over period of several minutes to several days without stirring.

The applicator body retains its shape and does not disintegrate and/or dissolve during the application time on the skin.

The water soluble filmogenic polymer is preferably selected from the group consisting of starch, pullulan, starch, cellulose, derivatives thereof, and blend thereof.

Advantageously, a water soluble polymer used in this invention is selected from the group consisting of cellulose and derivatives thereof. A good water soluble polymer for use in the present invention is cellulose ethers, notably water-soluble methylcellulose and especially hydroxypropyl methylcellulose (HPMC) polymers, for example such as those available for example under trade names METHOCEL K35, METHOCEL A15LV, METHOCEL Premium E5 LV and K35 LV from Dow chemical co.

According to one specific embodiment, the applicator body composition comprises or is consisting of clay, for example fuller's earth and/or Kaolin, and methylcellulose (HPMC) and water (residual water after drying).

Advantageously, the composition of applicator body may also comprise of at least one surfactant. Surfactant also imparts the flexibility to film but also contributes to cleansing of skin and therefore enhances the efficacy of clay. The surfactant may be selected from anionic, cationic, amphoteric and non-ionic surfactant. Non limiting examples of surfactants include alkyl sulfates, ethoxylated alkyl sulfates, betaines, taurates, sarcosinates. Specific examples include but are not limited to sodium laureth sulfate (SLES), cocamidopropyl betaine, sodium methyl cocoyl taurate, sodium cocoyl sarcosinate, disodium laureth sulfosuccinate, magnesium laureth sulfate, sodium laureth-8 sulfate and any mixture thereof.

In one embodiment, the applicator body comprising at least one surfactant releases surfactant during the contact of the applicator body with skin, which generates foam upon washing of face.

In one embodiment, the composition may additionally comprise one or more other topically acceptable ingredient.

In one embodiment, the composition may comprise at least one skin care agent and/or at least one cosmetically active ingredient.

Yet in another embodiment of the invention; the applicator body can also comprise at least one plasticizer which increases the flexibility and strength of applicator body and in some cases also provides skin conditioning benefits. Non-limiting examples of suitable plasticizers are glycerin, glycols such as ethylene glycol, propylene glycol, butylenes glycol and diethylene glycols, dextrins, sorbitols.

For example, the skin care agent and/or cosmetically active ingredient is selected from the group consisting of whitening agents, anti-oxidants, cleansing agents, free radical scavengers, moisturizers, skin tone altering agents, anti-acne agents, anti-aging agents, anti-wrinkle agents, anti-inflammatory agents, skin texture treatment agents, anti-perspirant agents, aesthetics, anti-bacterial agents, nourishing agents, sebum and moisture absorbers and any combination thereof.

The composition of said applicator body may contain other cosmetically ingredients such as cosmetically active ingredients which may be for example advantageous in addition to oil absorption. Other skin care agents or cosmetically active ingredients, such as for example whitening agents, anti-oxidants, cleansing agents, free radical scavengers, moisturizers, skin tone altering agents, anti-acne agents, anti-aging agents, anti-wrinkle-agents, anti-inflammatory agents, skin texture treatment agents, anti-perspirant agents, aesthetics, anti-bacterial agents, nourishing agents, sebum and or moisture absorbers and any combination thereof can be also used in the composition of applicator body to achieve one or more particular skin care benefits.

Non-limiting examples of skin whitening or brightening agents include, but not limited to, skin tone changing pigments, reflective particulate materials, soft focus agents and any mixture thereof. The skin tone changing pigments includes but not limited to, talc, mica, silica, titanium oxide, zinc oxide and or titanium oxide coated mica, borosilicates; hydrophobically modified zinc oxides; hydrophobically modified titanium di-oxides, composites of titanium-zinc oxides.

Example of anti-oxidants and or anti-inflammatory and/or cooling actives and/or sweat absorption actives to be used in the invention include, but not limited to, Vitamin C, Vitamin E, Derivatives of Vitamin C and Vitamin D, Lycopene, Carrotene, Lecithin, Rice bran lipids, Rice bran oils, menthol; yoghurt, silicated menthols, menthyl lactates; almond oil; peanut oil; sunflower oil; horse gram powder; olive oil; fumated or non-fumated silica, hydrophobically modified silica, hydrophylically silica and any mixture/composite thereof.

Advantageously, these actives are released to skin during the application of applicator body upon contact with skin.

The colour of applicator body will be mainly in the tone of colour of clay. But still desired colour can be achieved using the coloring additives in the composition. It can be inorganic or organic dyes and lakes which may or may not be surface treated. Non-limiting examples of such coloring additives include titanium dioxide, yellow iron oxide, red iron oxide, black iron oxide, and caramel, carmine, carbon black, D & C Red 7 Lake, FD & C Yellow 5 Lake, FD &C Yellow dye, FD & C Blue 1 dye.

The composition of applicator body comprises preferably from 1 to 50% clay; more preferably 1 to 20% and even more preferably 1-10% by mass of the total wet composition mass.

The amount of water soluble polymers in the composition of applicator body depends on the viscosity and/or water solubility of the polymer in aqueous solution. Generally it ranges from 1 to 20%; more preferably 1 to 10%; even more preferably from 2-5% by mass of the total wet composition mass.

The optional plasticizer ranges from 0.1% to about 20% by mass; more preferably 0.25% to about 10% and even more preferably from 2% to 5% by mass of total wet composition mass.

Proportions of components are such to produce a mud before drying.

Mud of the invention presents typically a viscosity of wet composition in mPa·s at 25.0 ranging from 3500 to 100000; more preferably from 3500 to 30000 and even more preferably from 4000 to 10000. The viscosity was measured using Prorheo rheomat R 180 at 25.0 using spindle M3.

In one embodiment, the applicator body is a skin mask.

In one embodiment, the applicator body is a face mask.

In one embodiment, the applicator body is a film or sheet.

In one embodiment, the applicator body is in the form of a single film or sheet.

In one embodiment, the applicator body is in the form of a two separate films or sheets.

In another embodiment, the applicator body is in the form of a three separate films or sheets.

Advantageously, a water soluble polymer is a filmogenic polymer.

In one embodiment, the applicator body presents a shape corresponding to the skin zone on which said applicator body has to be placed.

The film or sheet generally presents a thickness ranging from 0.01 mm to 1 mm; more preferably from 0.05 mm to 0.5 mm and even more preferably from 0.07 mm to 0.12 mm.

The applicator body has been advantageously developed by making complex of clay and water soluble polymer in order to get a dry film or sheet, which can be cut in the desired shape.

The most common format of applicator bodies distribution is folded, packed in a single use sachet. In some cases, applicator bodies are distributed in the format of a roll pack. Such a roll pack comprises a substrate roll with a plurality of applicator bodies of a predetermined shape, for example a plurality of face masks. A cosmetic product can be applied on such an applicator body after its detaching from the roll pack or any other packaging.

The body surface of the user is advantageously the skin of the user, in particular the skin located on a member of the user such as an arm or a leg, or preferably the skin of the face of the user. The shape of the applicator body is defined by its outer contour which depends on its targeted functionality and targeted zones.

One example of use of applicator body can be in the form of face mask, which can be cut in the shape of face with slits for eyes, mouth and nose. Applicator bodies are for example selected among face masks, wipes and pads.

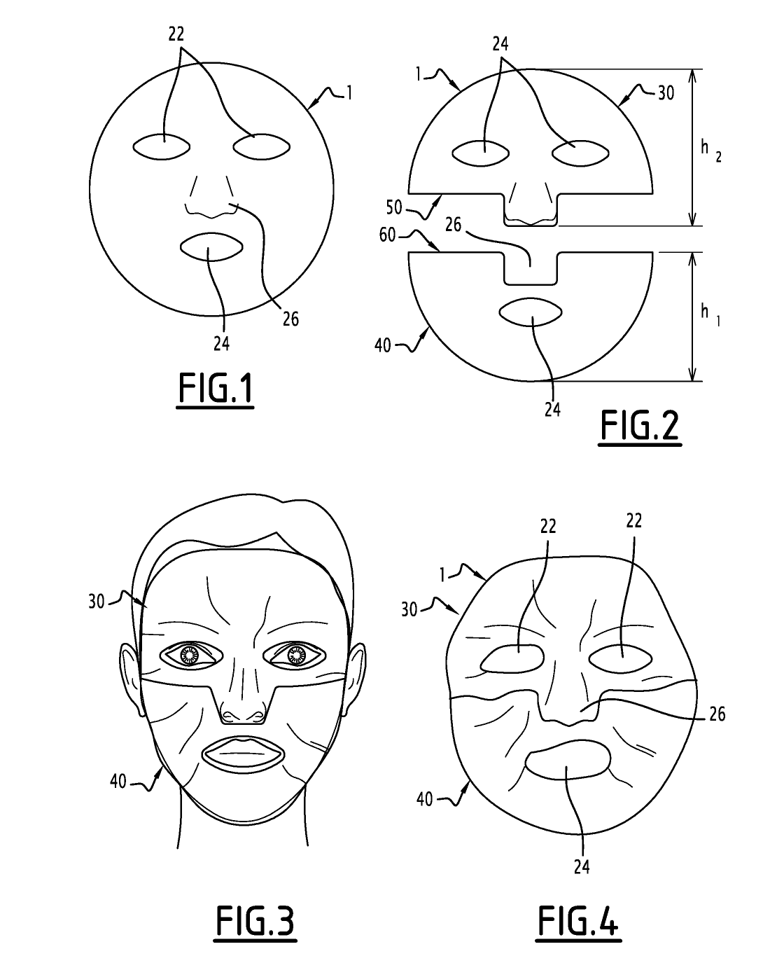

In a particular embodiment, in FIG. 1 applicator body, it has a rounded shape, for example a circular shape. Alternatively, the shape is polygonal. One example of proposed mask is shown in the figures. The mask shown in the figures is made up of complex of Fuller's earth with, Hydroxypropyl Methycellulose (HPMC) and SLES surfactant.

Advantageously, it comprises at least two through openings intended to be placed facing the eyes of a user, and another through opening intended to be placed facing the mouth of a user. The mask also comprises a slit delimiting a flapper intended to be pushed away by the nose of the user, to delimit a nose insertion through opening.

The invention is described with respect to FIGS. 1, 2, 3 and 4.

FIG. 1 represents a face mask made of one piece.

FIG. 2 represents a face mask made of two pieces, prior to use.

FIG. 3 represents an applicator body (face mask here) adhering on wet face. Such face mask of the invention becomes transparent on face. FIG. 3 represents a face mask during application onto the skin.

FIG. 4 represents an applicator body (face mask here) peeled off from face in 3-D face shape. FIG. 4 represents a face mask which has been used.

In a particular embodiment, the applicator body (1) is a face mask illustrated in FIG. 1. In this case, an outer contour of the mask defines an oval shape. In general the surface area of the oval shape is comprised between 0.25 cm² and 500 cm², preferably between 200 cm² and 450 cm².

Advantageously, the applicator body (1) comprises inner cavities. For the mask shown in FIG. 1, such inner cavities comprises two through openings (22) intended to be placed facing the eyes of the user, and another through opening (24) intended to be placed facing the mouth of the user.

The mask also comprises a slit (26) delimiting a flapper intended to be pushed away by the nose of the user, to delimit a nose insertion through opening.

In a variant, the oval shape of the face mask is completed with shapes facing the neck and/or ear hangings of the user.

In another variant, the applicator body (1) is formed into a patch or a garment having a volume, for example a sock, glove or sleeve.

Typically the applicator body of the invention is a one piece or two pieces face mask.

As shown in FIG. 2, the proposed mask can be cut in two pieces: Upper piece (30) and lower piece (40) in order to facilitate the easier application onto upper face and lower face, respectively. Such two pieces face mask presents an upper piece (30) intended to be placed on the upper part of the face and a lower piece (40) intended to be placed on the lower part of the face. The separation line or area (50, 60) of the two pieces is preferably made to delimit two pieces (30,40) having a height, wherein height is defined vertically from the chin to the nose (h1) for the lower piece (40) and from the nose to the forehead (h2) for the upper piece (30). According to one embodiment, h1 and h2 are essentially of the same distance. The separation line or area (50,60) separates the face mask in two pieces, extends preferably essentially horizontally and is located below the nose, preferably just below the nose. In one embodiment, the separation line delimits the slit delimiting nose insertion, opening (26).

The invention also relates to a method for preparing an applicator body according to the invention, said method comprising:
a) dispersing clay in an aqueous solution comprising a water soluble polymer;
b) obtaining an aqueous solution comprising dispersed clay.
The invention also relates to an aqueous solution or mud comprising in % by mass with respect to the total mass of the composition:
   from 1 to 50% of clay;
   from 1 to 20% of at least on water soluble filmogenic polymer;
   optionally from 1 to 30% of surfactant;
   optionally from 0.01 to 30% of other ingredients;
   the remaining is water and inevitable impurities.

The proposed applicator body may be prepared by dispersing the clay in a water soluble polymer matrix or gel which is solubilized or dispersed in an aqueous solution.

An aqueous solution is typically water.

In one embodiment, the aqueous solution comprises other components of the applicator body composition (other than clay and water soluble polymer).

In one embodiment, the method of preparation according to the invention comprises step c) of forming, preferably by casting, a film or sheet made with at least said aqueous solution comprising dispersed clay.

In one embodiment, the method comprises step d) of drying said film or sheet.

The dried film or sheet may be shaped according to the appropriate desired shape. Shaping may be done by cutting the film or sheet in the desired shape of applicator body.

Typically, the mixture of clay and water soluble polymer and optional components is preferably casted as film and then can be cut in the desired shape.

Film casting can be done using known techniques such as film casting process. The casting process may include preparation of complex of clay and water soluble polymer, and other optional components, metering of this formulation through film applicator, comprising of casting knife, uniform application of the aqueous solution comprising dispersed clay on casting conveyor and drying of film. The thickness of the film can be adjusted by clearance of film applicator.

The drying temperature of the film may in general range between 40° C. to 200° C.; more preferably between 40° C. to 100° C. and even more preferably between 40° C. to 70° C. The casted film may be separated from the casting conveyor and can be cut in the desired shape.

Advantageously, the applicator body of the invention dries quickly, typically in between 30 to 60 minutes.

Advantageously, the applicator body of the invention presents a viscosity adapted to film casting.

In one embodiment, the applicator body is placed onto a support, more preferably a film, and even more preferably a plastic film. The applicator body may be placed in between two supports, more preferably two film supports, and even more preferably two plastic films, such as PVC, PE or PP films. This aims to store said applicator body in good conditions. In a preferred embodiment, the applicator body is placed on such support or placed between such supports during casting.

Advantageously, the applicator body of the invention is easily removed from the support(s).

The present invention also relates to an applicator body comprising clay and a water soluble polymer for use as a cosmetic or skin care product.

The invention also relates to a method of cosmetic care comprising placing an applicator body according to the invention in contact with at least one skin zone of a human body.

Preferably, said method comprises wetting at least one skin zone of a human body prior to contacting said applicator body with this skin zone.

In one embodiment, said method comprises waiting a sufficient period of time, called “application time”, after placing said applicator body according to the invention in contact with at least one skin zone of a human body until said applicator body has naturally dried, and then removing said dried applicator body by peeling-off from said skin zone.

After the application time said applicator body can be easily peeled off from skin.

An applicator body according to the invention can be applied on the wet face as single layer and after natural drying on the skin, can be easily peel off as single layer.

Natural drying is obtained by just waiting without using any specific mechanical means or temperature increase.

The applicator body is typically applied on wet face for 15-20 minutes or till the drying of applicator body.

Natural mud is known to be very good oil absorber from the skin. Accordingly, one particular embodiment of the invention makes use of natural mud, such as Fuller's Earth, China clay, Ash, as clay in the composition of the applicator body.

In one embodiment, the applicator body is for treating or caring oily skin.

In particular the applicator body may be applied on wet surface of skin and absorbs impurities, sebum and excess oil from the skin.

In one embodiment, the applicator body is for mattifying skin.

In one embodiment, the applicator body is for relaxing cosmetic care.

In one embodiment, the applicator body is for refreshing skin care.

The functionality of the applicator body mainly depends on the activities of the active ingredients. Moisturisation, whitening and anti-aging are the most common functionalities for such an applicator body, especially face mask.

In one embodiment, the applicator body comprises a foaming agent. After peeling off from skin it may thus generate foam on skin, notably during rinsing with water.

This is one example of dual cosmetic functionality of peeling and cleansing.

Furthermore, other skincare active ingredients for cosmetic benefits such as for example whitening, anti-wrinkles etc. can be present or added, which may be released to the skin during the application time.

Applicator body comprising a surfactant advantageously facilitates a dual functionality of extracting the impurities and excess sebum through peeling of applicator body from skin and cleansing by leaving the cleansing substances on the skin.

Advantageously, the applicator body of the invention is easily removed from the skin, as single continuous piece, and if the applicator body is formed from multiple pieces, each piece is removed as a single continuous piece. More advantageously it is removed from skin without excessive stretching of skin, avoiding unwanted suffering of the user.

Advantageously, the applicator body of the invention is transparent or translucent on skin.

Some of the examples of compositions of applicator body of the invention are being given in the following examples:

In the examples all percentages (%) are in mass with respect to the total composition mass, except contrary indication, temperature is room temperature (20° C.), except contrary indication, and pressure is atmospheric pressure (101325 Pa), except contrary indication.

### EXAMPLES

### Example 1

### Composition of Applicator Body for Use as Clay Sheet Face Mask

Clay is dispersed in an aqueous solution containing a water soluble polymer according to the following proportions:

| Ingredient (INCI) | Trade name | Proportion (%) |
|---|---|---|
| Water | — | 94 |
| Solum Fullonum (Fuller's | Multani mitti | 4 |
| Earth) | | |
| Hydroxypropylmethyl | METHOCEL 4FM | 2 |
| cellulose | | |

The proposed composition was converted into a film of 0.08 mm thickness using film casting machine. This film can be cut into the shape of face mask and can be applied on wet face of user. It becomes dry in 15 minutes and then can be peeled off as single layer from the face.

### Example 2

### Composition Applicator Body for Use as Clay Sheet Face Mask Along with a Surfactant

The applicator body is used as as clay sheet face mask along with a surfactant in order to boost the efficacy of as well to make sheet more flexible.

Clay is dispersed in an aqueous solution containing a water soluble polymer (HPMC), and a surfactant according to the following proportions:

| Ingredient(INCI) | Trade name | Proportion (%) |
|---|---|---|
| Water | — | 88.75 |
| Solum Fullonum (Fuller's | Multani mitti | 4 |
| Earth) | | |
| Hydroxypropylmethyl | METHOCEL 4FM | 2 |
| cellulose (HPMC) | | |
| Sodium laureth sulfate | Galaxy LES 170 | 5 |
| (70% solution) | | |
| Fragrance | — | 0.25 |

Sodium laureth sulfate (INCI) = Sodium lauryl ether sulfate

The proposed composition in example 2 was converted into a film of 0.08 mm thickness using film casting machine. This film can be cut into the shape of face mask and can be applied on wet face of user. It becomes dry in 15 minutes and then can be peeled off as single layer from the face. After removal of mask, user needs to rub the face with water to feel the foam due to residual surfactant on the face. In this way proposed clay sheet facilitates dual functionality of extraction of impurities and cleansing.

### Example 3

### Composition Applicator Body for Use as Kaolin Sheet Face Mask

Kaolin is dispersed in an aqueous solution containing a water soluble polymer (HPMC), surfactant and fragrance, according to the following proportions:

| Ingredient(INCI) | Trade name | Proportion (%) |
|---|---|---|
| Water | — | 89.75 |
| Kaolin | Kaolin | 3 |
| Hydroxypropylmethyl | METHOCEL 4FM | 2 |
| cellulose (HPMC) | | |
| Sodium laureth sulfate | Galaxy LES 170 | 5 |
| (70% solution) | | |
| Fragrance | — | 0.25 |

### Example 4

### Evaluation of Cosmetic Care

Eight face masks made of two pieces (as shown in FIG. 2) were prepared using composition given in example 2. Eight users were asked to apply those masks on their wet face for 15 minutes. Sebum content and shine was evaluated after removal of mask and after 24 hours of the removal of mask using sebumeter, Samba. The criteria of selecting the users of the mask were the following:
8 Indian women subjects
Age: 25-40 years old;
Skin type: Oily skin;
Skin tone: fair, medium, dark—equal distribution;
No use of skincare products & face/eye make-up, on & one day before the test acquisition;
Absence of any clinical problems;
Absence of acne/pimples;
No facial or skin treatment done in past 2 weeks;

Users of mask could remove the mask in a single layer after 15 minutes of the application, which confirms its functionality as peel off product. After removal of mask, consumers rinsed off their face with water and felt slight foam on their face, it confirmed functionality as rinse off cleanser.

Reading of classical sebum was taken on the forehead of the consumers while shine was measured on forehead and chicks of the users.

The results regarding “shine” are expressed in % with respect to the “shine” of the user before application of the face mask.

The results regarding “sebum” are expressed in % with respect to “sebum” of the user before application of the face mask.

Results are average values for the panel of users.

During the application, face mask adopts the shape of user's face (FIG. 3) and is peeled off in 3-D shape (FIG. 4). After removal of the mask, it leaves surfactant on the skin, which generates light foam on the skin used as cleanser.

Results are as follows:

After immediate removal, the % shine evolution is as follows:

| | Shine (%) | Sebum (%) |
|---|---|---|
| After immediate removal | −7.25 | −90.66 |
| After 24 hrs from removal | +1.46 | −28.16 |

Results show that applicator body according to the invention reduces the sebum content and shine significantly after removal of applicator body.

Results show that the effect on sebum by application of applicator body according to the invention onto skin remains at least after 24 hours, which is considered a long lasting effect.

1. Applicator body, wherein said applicator body composition comprises at least one clay, at least one water soluble polymer and at least one surfactant, and wherein said applicator body presents a shape corresponding to the skin zone on which said applicator body has to be placed.

2. Applicator body according to claim 1, wherein said applicator body is a skin mask.

3. Applicator body according to claim 1, wherein said applicator body is a face mask.

4. Applicator body according to claim 1, wherein said applicator body is a film or sheet.

5. Applicator body according to claim 1, wherein said water soluble polymer is a filmogenic polymer.

6. (canceled)

7. Applicator body according to claim 1, wherein said composition additionally comprises one or more other topically acceptable ingredient.

8. Applicator body according to claim 1, wherein said composition comprises at least one skin care agent and/or at least one cosmetically active ingredient.

9. Applicator body according to claim 1, wherein said skin care agent and/or cosmetically active ingredient is selected from the group consisting of whitening agents, anti-oxidants, cleansing agents, free radical scavengers, moisturizers, skin tone altering agents, anti-acne agents, anti-aging agents, anti-wrinkle agents, anti-inflammatory agents, skin texture treatment agents, anti-perspirant agents, aesthetics, anti-bacterial agents, nourishing agents, sebum and moisture absorbers and any combination thereof.

10. (canceled)

11. Method for preparing an applicator body as defined in claim 1, wherein said method comprises:
a) dispersing clay in an aqueous solution comprising a water soluble polymer;
b) obtaining an aqueous solution comprising dispersed clay.

12. An aqueous solution or mud comprising in % by mass with respect to the total mass of the composition:
from 1 to 50% of clay;
from 1 to 20% of at least on water soluble filmogenic polymer;
optionally from 1 to 30% of surfactant;
optionally from 0.01 to 30% of other ingredients;
the remaining is water and inevitable impurities.

13. A method of cosmetic care comprising placing an applicator body as defined in claim 1 in contact with at least one skin zone of a human body.

14. The method of claim 13, wherein said method comprises wetting at least one skin zone of a human body prior to contacting said applicator body with this skin zone.

15. The method of claim 13, wherein said method comprises waiting a sufficient period of time, called “application time”, after placing said applicator body wherein the applicator body composition comprises at least one clay, at least one water soluble polymer and at least one surfactant, and wherein said applicator body presents a shape corresponding to the skin zone on which said applicator body has to be placed in contact with at least one skin zone of a human body until said applicator body has naturally dried, and then removing said dried applicator body by peeling-off from said skin zone.

16. Applicator body according to claim 2, wherein said applicator body is a face mask.

17. Applicator body according to claim 2, wherein said applicator body is a film or sheet.

18. Applicator body according to claim 3, wherein said applicator body is a film or sheet.

19. Applicator body according to claim 2, wherein said water soluble polymer is a filmogenic polymer.

20. Applicator body according to claim 3, wherein said water soluble polymer is a filmogenic polymer.

21. Applicator body according to claim 4, wherein said water soluble polymer is a filmogenic polymer.

22. Applicator body according to claim 2, wherein said composition additionally comprises one or more other topically acceptable ingredient.
